(19) United States
(12) Patent Application Publication
(10) Pub. No.: US 2026/0060542 A1
(43) Pub. Date: Mar. 5, 2026

(54) INFORMATION PROCESSING APPARATUS, TRANSFORMATION METHOD, STORAGE MEDIUM, AND INFORMATION PROCESSING SYSTEM

(71) Applicant: CANON KABUSHIKI KAISHA, Tokyo (JP)

(72) Inventors: ETSURO WADA, Kanagawa (JP); YOSHIHIKO IWASE, Kanagawa (JP); TSUKASA SAKO, Kanagawa (JP)

(21) Appl. No.: 19/381,719

(22) Filed: Nov. 6, 2025

Related U.S. Application Data
(63) Continuation of application No. PCT/JP2024/015515, filed on Apr. 19, 2024.

(30) Foreign Application Priority Data
May 11, 2023 (JP) 2023-078905

(51) Int. Cl. A61B 3/12; A61B 3/14; G06T 3/40; G06T 3/60; G06T 5/50

(52) U.S. Cl. CPC A61B 3/12; A61B 3/14; G06T 3/40; G06T 3/60; G06T 5/50; G06T 2207/20221

(57) ABSTRACT

An information processing apparatus according to the present disclosure includes a transformation unit configured to transform a first region and a second region in a fundus image so that an angle formed between a first straight line and a second straight line approaches 180 degrees, the first and second regions being defined by the first straight line and the second straight line, the second region being larger than the first region, the first straight line passing through a first feature portion related to a macula in the fundus image and being located on a temporal side, the second straight line passing through the first feature portion and a second feature portion related to an optic disc in the fundus image and being located on a nasal side.

### CROSS-REFERENCE TO RELATED APPLICATIONS

This application is a Continuation of International Patent Application No. PCT/JP2024/015515, filed Apr. 19, 2024, which claims the benefit of Japanese Patent Application No. 2023-078905, filed May 11, 2023, both of which are hereby incorporated by reference herein in their entirety.

### BACKGROUND

### Field of the Technology

The present disclosure relates to an information processing apparatus, a transformation method, a storage medium, and an information processing system.

### Description of the Related Art

As an ophthalmic apparatus, an Optical Coherence Tomography (OCT) apparatus for acquiring tomographic images of the fundus has become widely used. A physician diagnoses the presence or absence of ophthalmic diseases, such as glaucoma, by evaluating the retinal thickness (layer thickness) obtained from the tomographic images.

Japanese Patent Laid-Open No. 2022-38751 describes a method for evaluating the symmetry of layer thickness with respect to a straight line passing through the macular region and the optic disc region.

Here, the retinal thickness (layer thickness) has symmetry with respect to a straight line passing through the macular region and the optic disc region in a region on the nasal side of the macular region, but may lack symmetry in a region on the temporal side of the macular region, with respect to the straight line passing through the macular region and the optic disc region. In other words, a reference straight line for symmetry differs, so that, for example, the symmetry of the layer thickness can be appropriately evaluated in the nasal region, but may be inappropriately evaluated in the temporal region.

### SUMMARY

Thus, the present disclosure is directed to providing a method with which the symmetry of layer thickness in a nasal region and the symmetry of layer thickness in a temporal region can be appropriately evaluated.

According to an aspect of the present disclosure, an information processing apparatus includes a transformation unit configured to transform a first region and a second region in a fundus image so that an angle formed between a first straight line and a second straight line approaches 180 degrees, the first and second regions being defined by the first straight line and the second straight line, the second region being larger than the first region, the first straight line passing through a first feature portion related to a macula in the fundus image and being located on a temporal side, the second straight line passing through the first feature portion and a second feature portion related to an optic disc in the fundus image and being located on a nasal side.

Features of the present disclosure will become apparent from the following description of embodiments with reference to the attached drawings.

### BRIEF DESCRIPTION OF THE DRAWINGS

FIG. 1 is a block diagram of an information processing apparatus.

FIG. 2 is a flowchart illustrating an operation of a control unit.

FIGS. 3A to 3D are diagrams each illustrating features of a fundus image.

FIGS. 4A and 4B are diagrams each illustrating features of a fundus image.

FIGS. 5A and 5B are diagrams each illustrating features of a fundus image.

FIG. 6 is a diagram illustrating a method of transforming a fundus image.

FIG. 7 is a diagram illustrating a method of transforming a fundus image.

FIG. 8 is a diagram illustrating grids.

FIGS. 9A and 9B are diagrams illustrating a method of transforming a fundus image according to a second embodiment.

FIG. 10 is a diagram illustrating a method of transforming a fundus image according to the second embodiment.

FIG. 11 is a diagram illustrating a method of transforming a fundus image according to the second embodiment.

FIG. 12 is a diagram illustrating a method of transforming a fundus image.

FIG. 13 is a diagram illustrating fundus images on each of which a grid is superimposed.

FIG. 14 is a diagram illustrating fundus images on each of which a grid according to a third embodiment is superimposed.

FIGS. 15A and 15B are diagrams each illustrating features of a fundus image according to a fourth embodiment.

FIG. 16 is a flowchart illustrating an operation of the control unit according to the third embodiment.

FIG. 17 is a diagram illustrating a graphical user interface (GUI) displayed on a monitor.

FIG. 18 is a diagram illustrating a GUI displayed on the monitor.

FIG. 19 is a diagram illustrating a thickness map.

FIGS. 20A and 20B are diagrams illustrating a method of transforming a fundus image.

FIG. 21 is a diagram illustrating a method of transforming a fundus image.

### DESCRIPTION OF THE EMBODIMENTS

Illustrative embodiments for implementing the present disclosure will be described in detail below with reference to the accompanying drawings. In the drawings, the same reference numerals are used throughout the drawings to designate identical or functionally similar elements. In addition, in each drawing, some of the components, members, and processes that are not important for the description may be omitted.

Herein, the term “fundus image” refers to an image of the fundus obtained by an apparatus employing an imaging technique (modality), such as an optical coherence tomography (OCT) apparatus, a fundus camera, or a scanning laser ophthalmoscope (SLO). For example, the fundus image includes a tomographic image, a thickness map, an En Face image, and a projection image. Herein, the depth direction of the fundus is defined as a Z direction, and a plane perpendicular to the Z direction is defined as an XY plane.

Embodiments of the present disclosure will be described.

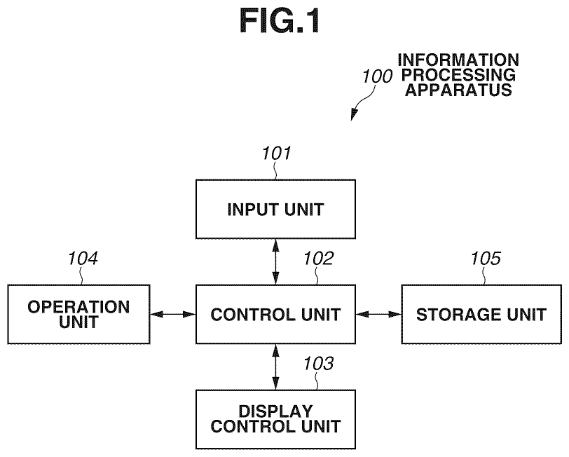

FIG. 1 is a block diagram of an information processing apparatus 100 according to an embodiment. The information processing apparatus 100 includes an input unit 101, a control unit 102, a display control unit 103, an operation unit 104, and a storage unit 105. The information processing apparatus 100 is, for example, a computer or a tablet terminal (portable information terminal). The information processing apparatus 100 does not necessarily have to be separate from an ophthalmic apparatus, such as an OCT apparatus. For example, the information processing apparatus 100 may be incorporated in an information processing system including an ophthalmic apparatus, such as an OCT apparatus.

The input unit 101 is, for example, a connector compatible with a universal serial bus (USB, registered trademark). Data is input to the input unit 101 from an ophthalmic apparatus, such as an OCT apparatus. The input unit 101 and the ophthalmic apparatus are connected by, for example, a cable. Here, the data to be input from the ophthalmic apparatus includes a fundus image. The data to be input from the ophthalmic apparatus may include data (such as an identification number, axial length of the eye (hereinafter, “axial length”), age, visual acuity, race, medical history, and whether the subject has high myopia) about a subject and an image of a medical record. In a case where an image of a medical record is included, data related to a subject may be extracted from the image of the medical record using a text mining technique or the like. Here, data may be input to the input unit 101 from an external apparatus, such as a server, in addition to the ophthalmic apparatus. The input unit 101 and the external apparatus may be directly connected by a cable or may be connected via a network. The connection between the input unit 101 and the external apparatus is not limited to a wired connection and may be a wireless connection. As long as the input unit 101 and the external apparatus can communicate with each other, the communication method is not limited.

The data input from the ophthalmic apparatus is output to the control unit 102.

The control unit 102 is, for example, a central processing unit (CPU). The control unit 102 performs image processing on the data input from the input unit 101. The image processing includes a transformation process for a fundus image. The control unit 102 operates according to programs stored in the storage unit 105. Here, the control unit 102 may be, in addition to the CPU, a micro processing unit (MPU), a graphics processing unit (GPU), or a field-programmable gate array (FPGA).

The display control unit 103 controls a display unit, such as a display. The display control unit 103 is, for example, one of the functions provided in the CPU. The display control unit 103 performs control to cause the display unit to display data input from the control unit 102. The data displayed on the display unit includes, for example, data related to a subject, various images, and estimation results related to ophthalmic diseases.

The operation unit 104 is, for example, a mouse or a keyboard. The operation unit 104 is operated by the operator. Information about the operation performed by the operator is input to the control unit 102.

The storage unit 105 is, for example, a non-volatile memory. The storage unit 105 stores a program for causing the control unit 102 to execute processing. The storage unit 105 stores an operating system (OS), drivers for peripheral devices, and programs for implementing various types of application software, including programs for performing processing described below. In addition, data to be used for various calculations is stored in the storage unit 105.

### [Features of Fundus Image]

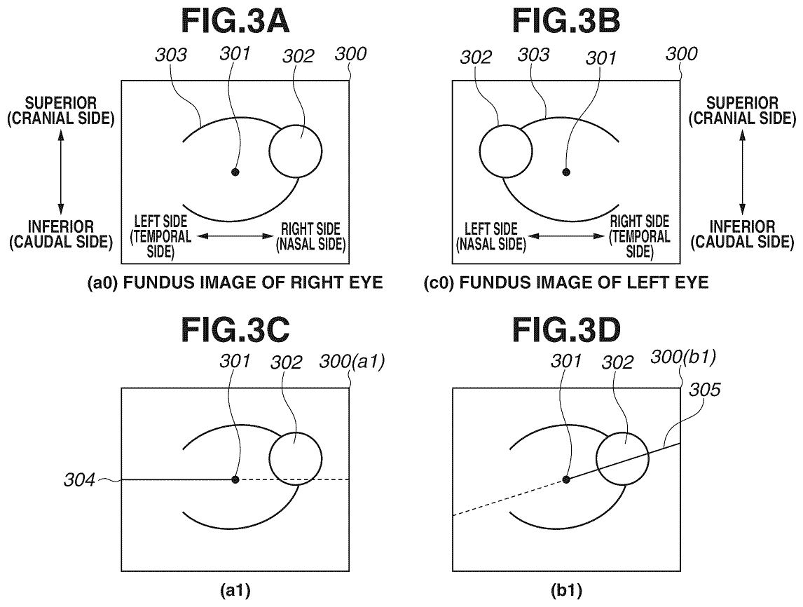

The features of a general fundus image will be initially described with reference to FIGS. 3A and 3B. FIG. 3A schematically illustrates a fundus image 300 of the right eye. The fundus image 300 includes a macular region 301, an optic disc 302, and blood vessels 303. Here, in the case of the fundus image 300 of the right eye, the right side of the fundus image 300 corresponds to the nasal side, and the left side of the fundus image 300 corresponds to the temporal side. The superior side of the fundus image 300 corresponds to the cranial side, and the inferior side of the fundus image 300 corresponds to the caudal side. FIG. 3B schematically illustrates a fundus image 300 of the left eye. As with the fundus image 300 of the right eye, the fundus image 300 of the left eye includes a macular region 301, an optic disc 302, and blood vessels 303. Here, for the fundus image 300 of the left eye, unlike that of the right eye, the right side of the fundus image 300 corresponds to the temporal side, and the left side of the fundus image 300 corresponds to the nasal side. Further, as with that of the right eye, the superior side of the fundus image 300 corresponds to the cranial side, and the inferior side of the fundus image 300 corresponds to the caudal side. Herein, the term “temporal side” refers to the left side in the fundus image of the right eye and the right side in the fundus image of the left eye. The term “nasal side” refers to the right side in the fundus image of the right eye and the left side in the fundus image of the left eye.

In imaging using the ophthalmic apparatus, the operator sets the position of a fixation target so that the macular region 301 of the eye under examination is located at a center of the fundus image. The operator then confirms that the eye under examination is in a state of directly gazing at the fixation target, then performs imaging. In a fundus image of the right eye captured in such a manner, in general, the macular region 301 is located substantially at the center of the fundus image 300, and the optic disc 302 is positioned on the superior nasal side of the macular region 301, as illustrated in the fundus image 300 of FIG. 3A.

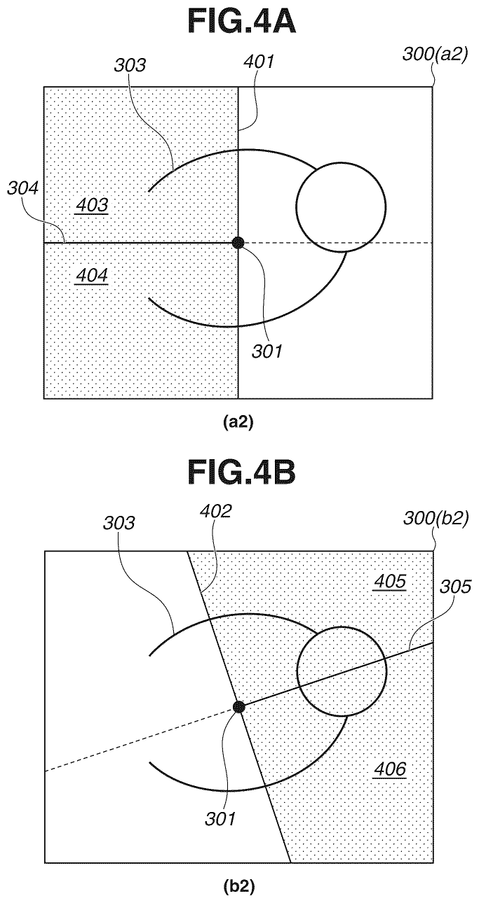

Characteristics of general retinal layers will now be described with reference to FIGS. 3A to 3D and 4A and 4B. Here, the term “retinal layer” refers to a retinal nerve fiber layer, a ganglion cell layer, an inner plexiform layer, and other layers.

A fundus image 300 (a1) in FIG. 3C illustrates a straight line 304 extending from the macular region 301 towards the nasal side and the temporal side. The straight line 304 extends substantially horizontally with respect to the fundus image 300 (a1). The portion of the straight line 304 extending from the macular region 301 toward the temporal side is illustrated as a solid line. The portion of the straight line 304 extending from the macular region 301 toward the nasal side is illustrated as a broken line. In addition, in a fundus image 300 (a2) of FIG. 4A, a straight line 401 passing through a macular region 301 and orthogonal to a straight line 304 is illustrated. It is known that, among the four regions segmented by the straight lines 304 and 401, the thickness (layer thickness) of the retinal layers in a region 403 located on the superior temporal side and the layer thickness of a region 404 located on the inferior temporal side tend to be symmetrical with respect to the straight line 304.

In a fundus image 300 (b1) of FIG. 3D, a straight line 305 connecting a macular region 301 and an optic disc 302 is illustrated. The portion of the straight line 305 extending from the macular region 301 toward the nasal side is illustrated as a solid line. The portion of the straight line 305 extending from the macular region 301 toward the temporal side is illustrated as a broken line. In a fundus image 300 (b2) of FIG. 4B, a straight line 402 passing through a macular region 301 and orthogonal to a straight line 305 is illustrated. It is known that, among the four regions segmented by the straight lines 305 and 402, the thickness (layer thickness) of the retinal layers in a region 405 located on the superior nasal side and the layer thickness of a region 406 located on the inferior nasal side tend to be symmetrical with respect to the straight line 305.

In a typically known method for evaluating symmetry, a fundus image 300 is geometrically transformed (e.g., by enlarging, reducing in size, rotating, translating, or performing an affine transformation) so that the straight line 305 connecting the macular region 301 and the optic disc 302 becomes substantially horizontal, and the symmetry of the layer thickness is then evaluated using the geometrically transformed fundus image. However, in the geometrically transformed fundus image, while the symmetry between the layer thickness of the region 405 on the superior nasal side and the layer thickness of the region 406 on the inferior nasal side can be appropriately evaluated, the symmetry between the layer thickness of the region 403 on the superior temporal side and the layer thickness of the region 404 on the inferior temporal side cannot be appropriately evaluated because the straight line 304 does not become substantially horizontal.

In the present embodiment, a transformation method will be described that enables appropriate evaluation of the symmetry between the layer thickness of the region 405 on the superior nasal side and the layer thickness of the region 406 on the inferior nasal side, as well as the symmetry between the layer thickness of the region 403 on the superior temporal side and the layer thickness of the region 404 on the inferior temporal side.

### First Embodiment

In the present embodiment, a description will be provided mainly using fundus images of the right eye; however the description is also applicable to fundus images of the left eye.

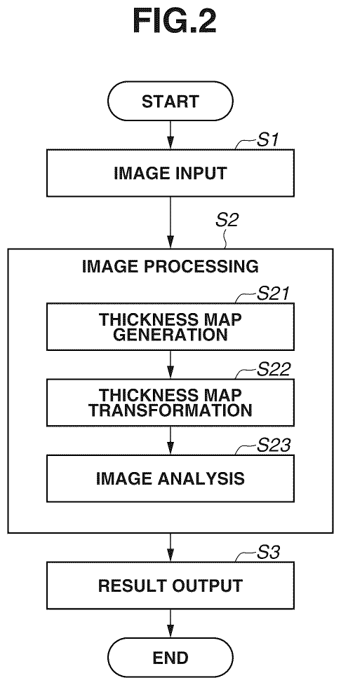

FIG. 2 is a flowchart illustrating the operation of the control unit 102 according to the first embodiment.

### [Step S1: Image Input]

In step S1, a tomographic image of the fundus obtained with an OCT apparatus is input from the input unit 101 to the control unit 102. Here, the term “tomographic image” includes images captured at a plurality of B-scan positions. Through the operation of step S1, the control unit 102 can acquire three-dimensional data on the fundus of the eye under examination. Here, A-scan refers to scanning at a single point on the eye under examination to obtain tomographic information. A B-scan refers to performing A-scans a plurality of times in a certain transverse direction (main scanning direction) to obtain two-dimensional tomographic information in the transverse and depth directions of the eye under examination. In the present embodiment, tomographic images of both the right and left eyes of the same subject are provided as input. In a case where there is a difference in axial length between the right and left eyes, a correction process may be performed.

### [Step S2: Image Processing]

In step S2, the control unit 102 performs image processing on a tomographic image input from the input unit 101. Step S2 includes the following processes: step S21 (thickness map generation), step S22 (thickness map transformation), and step S23 (image analysis).

### [Step S21: Thickness Map Generation]

In step S21, the control unit 102 generates a thickness map using the tomographic image input in step S1. The term “thickness map” refers to a map (map image) that represents, by brightness values or the like, the thickness (layer thickness) of anatomically defined observation target layers within the retina at any coordinate (i.e., XY coordinate) on an XY plane perpendicular to the depth direction (Z direction) of the eye under examination. An example of the observation target layers includes three layers, namely, the nerve fiber layer (NFL), the ganglion cell layer (GCL), and the inner plexiform layer (IPL). The layer thicknesses of these layers are summed, thereby generating the thickness map. Alternatively, the thickness map may be generated based on the layer thickness of only the NFL, or by selecting any layer and generating a thickness map based on the layer thickness of the selected layer. As a method for selecting a certain layer, the boundary between layers may be specified by manually drawing a line on the tomographic image, or selected from the boundary between layers obtained through a segmentation process. The segmentation process refers to a process of segmenting the tomographic image into individual layers. Specifically, the layer structure is extracted from the tomographic image of the eye under examination, the extracted layer structure is segmented into individual layers, and the thickness of each layer is calculated. Any available method may be used for the segmentation process and for calculating the thickness.

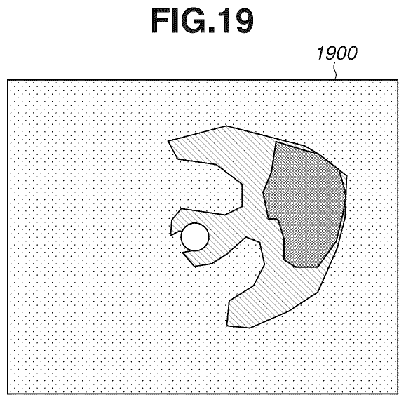

In the thickness map, the layer thickness at any given coordinate (XY coordinate) may be displayed using pseudo colors. Alternatively, in the thickness map, the layer thickness at any given coordinate (XY coordinate) may be displayed using grayscale luminance values. Further alternatively, a thickness map displayed in pseudo colors and a thickness map displayed in grayscale luminance values may be displayed side by side. Still alternatively, a thickness map displayed in pseudo colors and a thickness map displayed in grayscale luminance values may be displayed in a manner that allows switching between them. FIG. 19 illustrates an example of a thickness map 1900 corresponding to the fundus images 300 illustrated in FIG. 3A. In the thickness map 1900, the layer thickness is displayed using grayscale luminance values. In FIG. 19, a lighter color (higher luminance value) indicates that the layer thickness is thinner.

Here, the control unit 102 may perform a size reduction process or a trimming process on the input tomographic image before generating the thickness map. This method can be expected to reduce the processing load.

Next, the control unit 102 performs a correction process on the generated thickness map. Examples of the correction process are described below. As one example, the correction process may include a process (an error correction process) for correcting luminance values in a region that cannot be used for diagnosis in the thickness map. For example, in a case where an error occurs in the layer segmentation process, the thickness at the position exhibits an anomalous value and appears as blown out or crushed blacks in the thickness map. The control unit 102 determines a region having an anomalous value to be a region that cannot be used for diagnosis. The control unit 102 then performs a process of replacing the luminance values of the region that cannot be used for diagnosis with an average value of luminance values in the surrounding regions. Alternatively, the control unit 102 may remove the region that cannot be used for diagnosis from the thickness map. In addition, the control unit 102 may similarly replace or remove the luminance values of regions that are assumed to be unnecessary for diagnosis, such as within the optic disc.

In addition, in a case where the image data input from the input unit 101 is determined to have low image quality and be unusable for diagnosis, the control unit 102 can cause the display control unit 103 to display a message prompting re-imaging. This determination may be made visually by the operator. Alternatively, the determination may be made based on a rule-based approach using factors, such as image brightness and segmentation success rate. Still alternatively, the determination may be made using a method based on machine learning.

In generating the thickness map, the value of the layer thickness may be corrected based on axial length data on the eye under examination. Here, the axial length data on the eye under examination may be acquired in step S1 or may be input by the operator. In a case where the axial length of the eye under examination is longer than those of eyes in a normal-eye database, the retina tends to become stretched, which may result in a decrease in overall layer thickness. Thus, the control unit 102 can correct the thickness map to reduce the effect of the layer thickness that varies depending on the axial length. The correction of the layer thickness according to the axial length may be performed using any available method. Such correction enables the distinction between a case where the layer thickness is reduced due to a lesion and a case where the layer thickness is reduced from a normal state. In addition, the imaging range of the fundus relative to a scan angle varies depending on the axial length, so that the control unit 102 may perform a process of correcting the scale of the thickness map. Further, for the subject having high myopia, the axial length tends to be long. Thus, for the subject having high myopia, the control unit 102 may perform a similar correction on the thickness map. The axial length of the eye under examination and whether the subject is highly myopic may be determined based on input data relating to the subject.

While the thickness map is mainly used in the description of the present embodiment, the present disclosure is not limited to the thickness map, and a difference map based on a difference obtained by comparing the layer thickness with those of eyes in a normal eye database, a vessel density map generated using an OCT angiography image, or other types of maps may be used.

### [Step S22: Thickness Map Transformation]

In the step S22, the control unit 102 transforms the thickness map generated in step S21. In the present embodiment, an example will be described in which an En Face image is generated from the tomographic image input via the input unit 101, and the generated En Face image is transformed, thus transforming the thickness map. Here, the term “En Face image” refers to an image generated by projecting the tomographic image in the depth direction (Z direction). Generally, in a case where both an En Face image and a thickness map are generated from the same tomographic image, transforming the En Face image also results in a corresponding transformation of the thickness map. The control unit 102 configured to transform the thickness map is an example of a transformation unit.

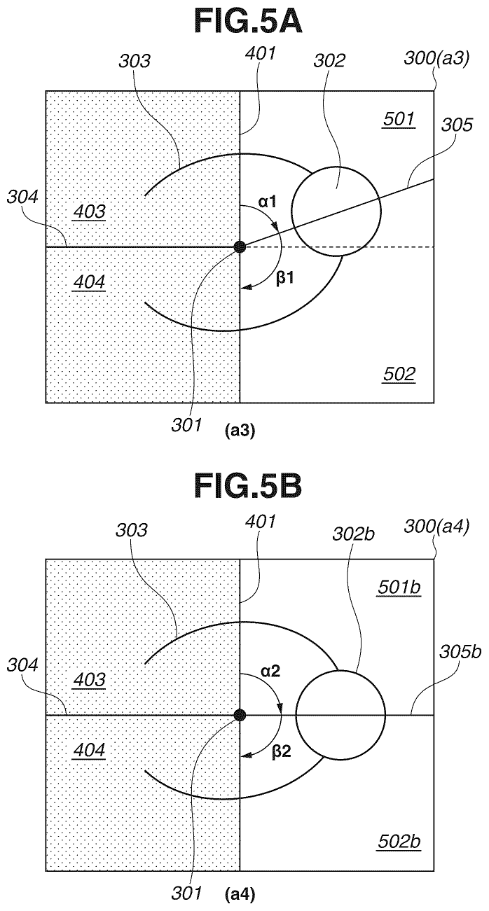

A specific transformation method will be described with reference to FIGS. 5A and 5B.

Initially, a fundus image is segmented.

A fundus image 300 (a3) in FIG. 5A is assumed to be an En Face image generated from a tomographic image input via the input unit 101. In addition to the straight lines 304 and 401 illustrated in the fundus image 300 (a2) of FIG. 4A, the fundus image 300 (a3) further includes a straight line 305 connecting a macular region 301 and an optic disc region 302. Among the regions segmented by the straight lines 401 and 305, the region located on the superior nasal side is defined as a region 501. The angle formed between the straight lines 401 and 305 in the region 501 is defined as α1. Among the regions segmented by the straight lines 401 and 305, the region located on the inferior nasal side is defined as a region 502. The angle formed between the straight lines 401 and 305 in the region 502 is defined as β1. Here, the positions of the macular region 301 and the optic disc region 302 may be set based on an instruction from the operator or may be automatically set through a fundus image analysis. The straight lines 401 and 305 do not necessarily have to pass through the macular region 301 itself and may pass through a peripheral portion of the macular region 301. Similarly, the straight line 305 does not necessarily have to pass through the optic disc region 302 itself and may pass through a peripheral portion of the optic disc region 302. The macular region 301 or a peripheral portion thereof is an example of a first feature portion relating to the macula. The optic disc region 302 or a peripheral portion thereof is an example of a second feature portion relating to the optic disc. The solid portion of the straight line 304 is an example of a first straight line. The straight line 305 is an example of a second straight line.

Any straight lines that substitute for the straight lines 401 and 305 may be manually set, and the values of the angles α1 and β1 may also be manually specified. The method of specifying the lines and values is not limited, and the lines and values may be directly specified on the fundus image, may be specified by input of coordinates or numerical values using a user interface (not illustrated), or may be specified by selection from preset numerical values.

The fundus image is then transformed.

In the present embodiment, an example will be described in which the regions 403 and 404 (hatched regions in FIGS. 5A and 5B) on the temporal side of the fundus image are not transformed, and the regions 501 and 502 on the nasal side are transformed. A fundus image 300 (a4) is an image resulting from the transformation of the fundus image 300 (a3) performed by the control unit 102. Regions 501b and 502b in the fundus image 300 (a4) are regions resulting from the transformation of the regions 501 and 502 performed by the control unit 102. In the present embodiment, the control unit 102 enlarges the region 501 and reduces the size of the region 502 so that the angle formed between the straight lines 304 and 305 (i.e., 90 degrees+α1) approaches 180 degrees. In other words, the control unit 102 transforms the regions with the macular region 301 serving as the center so that the straight line 305 approaches the broken-line portion of the straight line 304 . . . . The straight line 305 after transformation corresponds to a straight line 305b in the fundus image 300 (a4). The fundus image 300 (a4) illustrates an example in which, as a result of the transformation, the angle formed between the straight lines 304 and 305b becomes 90 degrees+α2. The angle formed between the straight lines 304 and 305b (90 degrees+α2) does not necessarily have to become 180 degrees as a result of the transformation. The effect of the present disclosure can be achieved as long as the angle formed between the straight lines 304 and 305b (90 degrees+α2) is closer to 180 degrees than the angle formed between the straight lines 304 and 305 (90 degrees+α1).

As a result of this transformation, the region 501 on the superior nasal side is transformed to be stretched in the circumferential direction and becomes the region 501b. In other words, the region 501 on the superior nasal side is enlarged. In addition, the angle α1 is transformed into the angle α2 (where α2>α1). In contrast, the region 502 on the inferior nasal side is transformed in a direction in which the region 502 is compressed in the circumferential direction and becomes the region 502b. Specifically, the region 502 on the inferior nasal side is reduced in size. In addition, the angle β1 is transformed into an angle β2 (where β2<β1). The region including the regions 403 and 501 is an example of a first region. The first region is defined by the straight lines 304 and 305 in the fundus image. The region including the regions 404 and 502 is an example of a second region. The second region is also defined by the straight lines 304 and 305 in the fundus image.

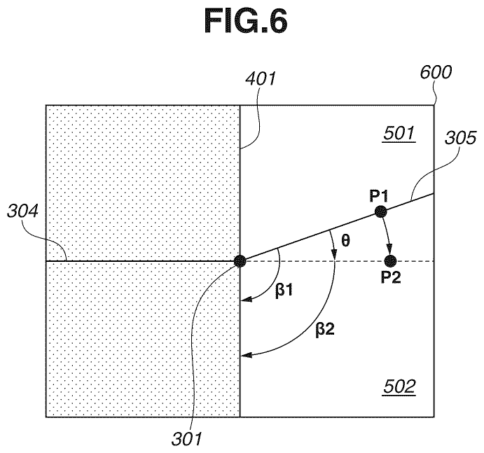

A more specific transformation method will be described with reference to FIG. 6. In a coordinate system in which a macular region 301 is the origin, a straight line 304 is the x-axis, and a straight line 401 is the y-axis, an angle θ (β1−β2) formed between a straight line 305 and the x-axis is obtained, and based on the angle θ, a given point P1 on the straight line 305 is transformed into a corresponding point P2 on the x-axis. The method for obtaining the coordinates of point P2 is not limited, but the coordinates of point P2 can easily be obtained by using a formula of a rotated coordinate system, with the rotation angle of point P1 set to θ.

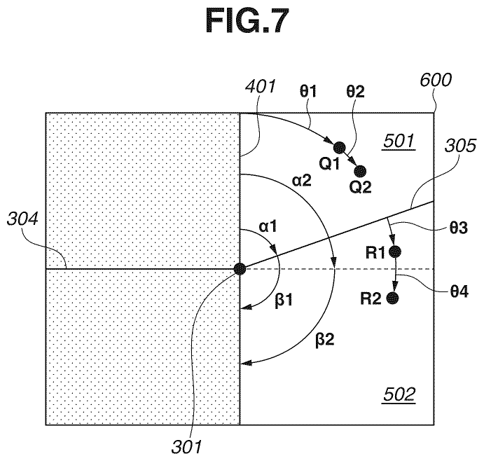

Similarly, a method for transforming the region 501 on the superior nasal side into the region 501b, and the region 502 on the inferior nasal side into the region 502b will be described with reference to FIG. 7. When θ1 is an angle formed between the y-axis and an unillustrated straight line connecting a given point Q1 on a region 501 and a macula region 301, a rotation angle θ2 of a point Q2 corresponding to the point Q1 can be obtained using the following equation:

$$θ 2 = θ 1 ( α 2 / α 1 ) - θ 1 = θ 1 ( ( α 2 / α 1 ) - 1 ) .$$

Further, when θ3 is an angle formed between the straight line 305 and an unillustrated straight line connecting a given point R1 on the region 502 and the macula region 301, a rotation angle θ4 of a point R2 corresponding to the point R1 can be obtained using the following equation:

$$θ 4 = θ 3 ( β 2 / β 1 ) + ( β 1 - β 2 - θ 3 ) = θ 3 ( ( β 2 / β 1 ) - 1 ) + ( β 1 + β 2 ) .$$

By applying a similar transformation process to each XY coordinate of the regions 501 and 502 as described above, the regions 501 and 502 can be transformed into the regions 501b and 502b. In the regions 501b and 502b, resulting from the transformation, in a case where there is no source point S for transformation (not illustrated) corresponding to a given point S′ (not illustrated) in the orthogonal coordinate system (i.e., no value is assigned), data on adjacent coordinates may be used as is, or interpolation may be performed using nearby data. Further, in a case where no source point for transformation is present, for example, at an edge of the image, and interpolation from adjacent points is not possible, a display may be provided to indicate the absence of data, for example, by blacking out or applying hatching. In a case where coordinates after transformation at an edge of an image falls outside the image, the image size may be increased so that no information is lost. In addition, in a case where a plurality of source points (e.g., points S1 and S2) for transformation corresponds to the certain point S′, either the data on the point S1 or S2 may be used for the point S′, or the data on the points S1 and S2 may be used after processing, such as averaging or other suitable processing.

In this example, the points P2, Q2, and R2 have been described as being transformed using a rotational coordinate system centered on the macular region 301, but a similar transformation process may be applied using a point other than the macular region 301 as the center, and each straight line used in the description may be replaced with a straight line that does not pass through the macular region 301.

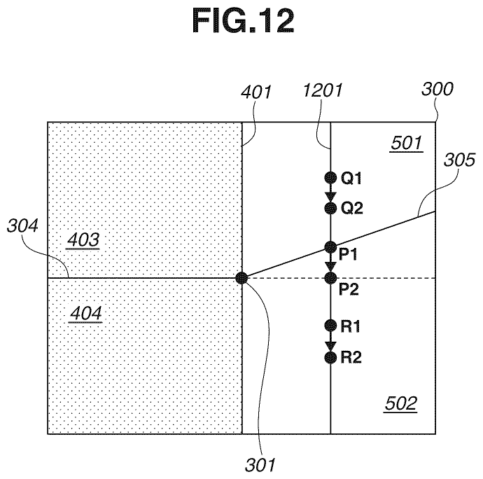

Coordinates may be transformed using another transformation method without using the rotation angles α1 and β1. For example, transformation may be performed using only the y-coordinate component in the orthogonal coordinate system. For example, from a given point P1 on a straight line 305 of a fundus image 300 in FIG. 12, a straight line 1201 orthogonal to a dashed portion of a straight line 304 is extended, and transformation is performed along the straight line 1201 such that the point of intersection between the straight line 1201 and the straight line 304 becomes the point P2. Similarly, the regions 501 and 502 may be transformed so that the given point Q1 on the region 501 and a given point R1 on the region 502 are positioned at the points Q2 and R2, respectively, along the straight line 1201. In this example, the description has been provided using the straight line 1201 orthogonal to the straight line 304, but the straight line 1201 may be any straight line having a certain inclination with respect to the straight line 304.

Weighting may also be performed using characteristics, such as the axial length and retinal distortion, during coordinate transformation. In this example, the region 403 on the superior temporal side and the region 404 on the inferior temporal side have been described on the assumption that the regions 403 and 404 are symmetrical with respect to the straight line 304, which extends substantially horizontally from the macular region 301. Alternatively, the entire fundus image may rotate depending on the imaging conditions, and the regions 403 and 404 are not necessarily symmetrical with respect to the straight line 304. In such a case, an unillustrated straight line 304′ having an angle such that the difference between the regions 403 and 404 becomes small may be used. Here, the entire fundus image 300 can be rotated so that the straight line 304′ becomes substantially horizontal, and then the transformation process can be performed.

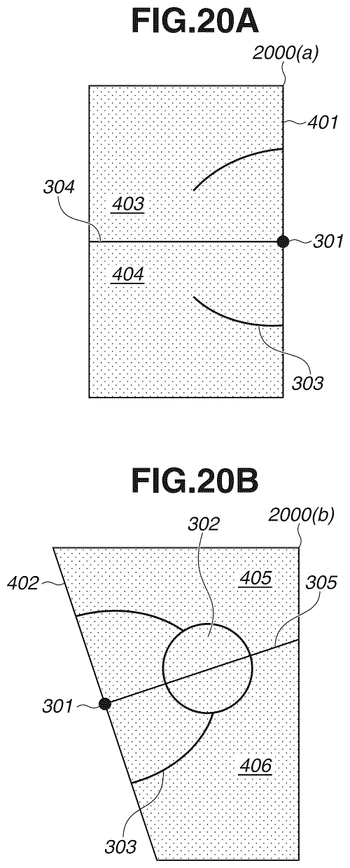

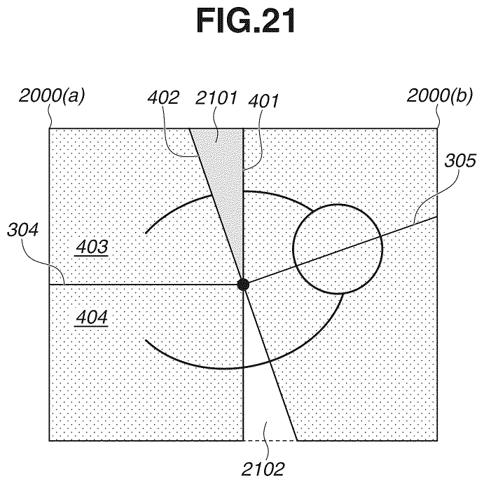

The fundus image 300 may also be segmented into a plurality of partial images. For example, FIGS. 20A and 20B illustrate partial images 2000(a) and 2000(b) obtained by cutting out the hatched regions of the respective fundus images 300 (a2) and 300 (b2) in FIGS. 4A and 4B, respectively. In this case, a partial region of the original fundus image 300 may be included in both partial images 2000(a) and 2000(b), or may be included in neither the partial image 2000(a) nor the partial image 2000(b). For example, FIG. 21 illustrates a diagram in which the partial images 2000(a) and 2000(b) are superimposed. In this case, a dark gray region 2101 is included in both the partial images 2000(a) and 2000(b), and a white region 2102 is included in neither the partial image 2000(a) nor the partial image 2000(b). Here, any transformation process may be applied to each of the partial images 2000(a) and 2000(b), and the subsequent step S23 (image analysis) may be performed. The partial images 2000(a) and 2000(b) do not necessarily have to be transformed, and subsequent steps may be performed without execution of the transformation process. In a case where a plurality of analysis results is obtained through the subsequent step S23 (image analysis) performed on each of the partial images 2000(a) or 2000(b), the plurality of analysis results may be displayed individually, or a single result obtained through integration of the plurality of analysis results may be displayed.

In the description of this example, an En Face image or a projection image is transformed to also transform the corresponding thickness map, but only the thickness map may be transformed with the En Face image or the projection image not being transformed. Further, instead of an En Face image, a fundus photograph or a fundus image acquired by another device, such as an SLO, may be used. In such a case, a fundus photograph or a fundus image obtained with an SLO or another device before transformation may be aligned with a thickness map in advance, and a similar transformation process may be applied to the thickness map in accordance with the transformation of the fundus photograph or the fundus image obtained with the SLO or the other device.

The object to be transformed is not limited to a two-dimensional image, and OCT three-dimensional data may also be transformed. The two-dimensional images described in the present embodiment (e.g., an En Face image, a projection image, and a thickness map) are generated by projecting information contained in an A-scan in OCT imaging in the Z direction. Thus, if coordinate transformation of each XY coordinate in the two-dimensional image can be calculated, the coordinate transformation of the A-scan in the three-dimensional OCT data can also be performed. By changing the XY coordinates assigned to each A-scan, transformed OCT three-dimensional data is constructed. Then, using the transformed three-dimensional OCT data, any transformed two-dimensional image (an En Face image, a projection image, a thickness map) can be generated.

### [Step S23: Image Analysis]

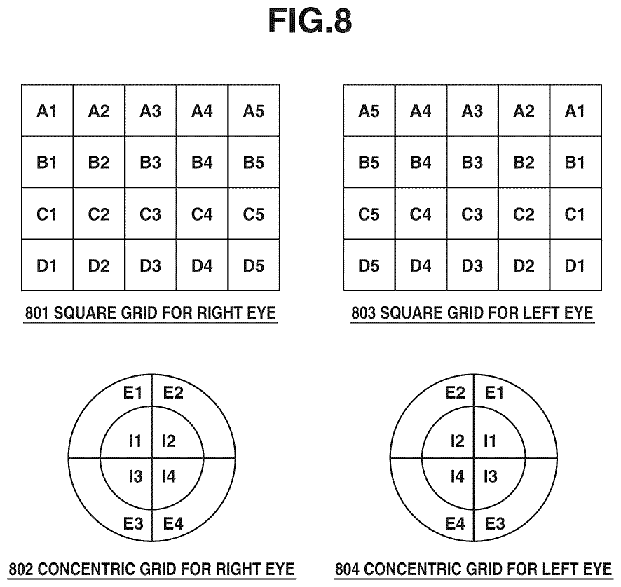

In step S23, the control unit 102 analyzes the fundus image using the transformed fundus image and the thickness map obtained in step S22. Specifically, the control unit 102 superimposes a grid that defines a plurality of evaluation regions in the fundus image, along with evaluation indices, onto the transformed fundus image 300 (a4). As an example of the grid, FIG. 8 illustrates a square grid 801 and a concentric circular grid 802 for the right eye. For each of a plurality of evaluation regions (e.g., A1, B1, etc.) included in the grids, the average or median value of pixel values of the thickness map within the corresponding evaluation region is assigned as an evaluation index. Here, symbols are assigned to the evaluation regions for convenience. For example, in the square grid 801 including 4 rows and 5 columns, the rows are labeled A to D from top to bottom, and the columns are labeled 1 to 5 from the temporal side. Each evaluation region is assigned a symbol, namely, A1 to E5. In the concentric circular grid 802, which is segmented into four inner and four outer regions, the inner cells are labeled “I” and the outer cells “E”. The regions are numbered as follows: superior-temporal as 1, superior-nasal as 2, inferior-temporal as 3, and inferior-nasal as 4. Each evaluation region is assigned a symbol, namely, I1 to I4 and E1 to E4. Meanwhile, the symbols for a square grid 803 and a concentric circular grid 804 for the left eye are defined as left-right symmetrical counterparts of those for the right eye. The average or median value of pixel values of the thickness map within each evaluation region is an example of an evaluation index generated using thickness information about the fundus. The control unit 102 serves as an example of an image generation unit configured to generate a fundus image on which the grid and evaluation indices are superimposed. The evaluation indices may also be standard deviation or variance of the thickness map.

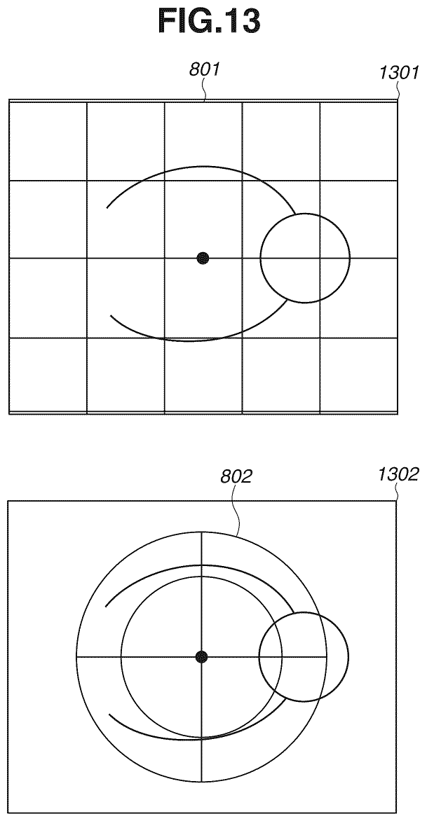

FIG. 13 illustrates fundus images 1301 and 1302, in which the square grid 801 and the concentric circular grid 802, respectively, are superimposed on the fundus image 300 (a4) of FIG. 5B. These fundus images 1301 and 1302 are examples of a second fundus image. Here, the symmetry of each evaluation region is considered. For the square grid 801 superimposed on the fundus image 1301, there is a tendency of symmetry between evaluation regions in the same column of rows A and D (e.g., there is a tendency of symmetry between cells A1 and D1), and also between the same column of rows B and C (e.g., there is a tendency of symmetry between cells B1 and C1). For the concentric circular grid 802 superimposed on the fundus image 1302, there is a tendency of symmetry between cells I1 and I3, between cells I2 and I4, between cells E1 and E3, and between cells E2 and E4. Thus, the operator evaluates the symmetry of the fundus image by comparing the cells that have a symmetrical correspondence. There are various possible methods for evaluating symmetry. For example, one method involves normalizing the values of the evaluation regions using the average value, and then calculating the difference between an evaluation region on the superior side (e.g., A1) and an evaluation region on the inferior side (e.g., D1) in evaluation regions having symmetrical correspondence, by subtracting the value (thickness) of the inferior evaluation region from the value (thickness) of the superior evaluation region, thus obtaining differential information. If the difference indicated by the obtained difference information is denoted as R and a reference value is denoted as N, then:
if R<−N (Group 1), the superior evaluation region is thinner than the inferior evaluation region;
if −N≤R≤N (Group 2), there is no difference between the superior and inferior evaluation regions (i.e., symmetry); and
if N<R (Group 3), the inferior evaluation region is thinner than the superior evaluation region.
   Furthermore, in a case where many evaluation regions fall into Group 1 or Group 3, it can be evaluated that symmetry is disrupted. The method for evaluating symmetry is not limited to this; for example, the sum of squared differences may be used, or weight(s) may be assigned to each cell. The reference value N may be set manually, or may be set automatically based on the average value of the evaluation regions. The difference R indicated by the obtained difference information or information about Groups 1 to 3 into which the evaluation regions are classified based on the reference value N may also be superimposed on the fundus image 1301 or 1302. These fundus images 1301 and 1302 having been subjected to the superimposition are examples of a third fundus image.

In the present example, symmetry is determined using the grids and the thickness map; however, symmetry may also be analyzed by assigning other types of analysis values to a map, such as a difference map obtained through comparison using a normal-eye database, or a vessel density map derived from an OCT angiography image. In comparing a transformed fundus image with another fundus image or those in a normal-eye database, a transformation process similar to that in step S22 may be applied to the comparison target fundus image or fundus image(s) in the normal-eye database, and then comparison may be performed. Alternatively, a difference map or the like may be generated before the transformation in step S22, and then the difference map or the like may be transformed in step S22.

### [Step S3: Display]

In step S3, the control unit 102 outputs the fundus image generated in step S23 to the display control unit 103. The image to be output here may be the fundus image generated in step S23, or an image generated by freely combining a transformed fundus image, a thickness map, a grid, and analysis results. In a case where a fundus image is displayed on a monitor, a pre-transformation fundus image and a thickness map as well as a post-transformation fundus image and a thickness map may be displayed side-by-side or in a switchable manner therebetween. For comparison or follow-up observation, in a case where fundus images of right and left eyes or a plurality of images of the same eye are/is to be displayed side-by-side or in a switchable manner, a similar transformation process may be applied to all the fundus images and then the processed fundus images are displayed. The fundus image(s) may be provided not only to a monitor but also output to a printer or other output device, or to the storage unit 105 or an external storage medium (not illustrated).

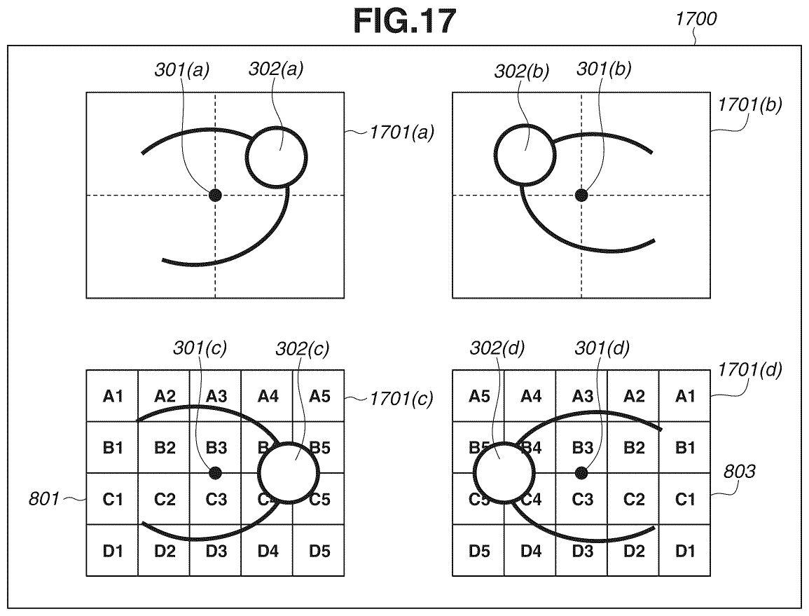

FIG. 17 illustrates an example of a graphical user interface (GUI) displaying fundus images of both eyes on a monitor 1700. Initially, for a fundus image 1701(a) for the right eye, the coordinates of a macular region 301 (a) and an optic disc region 302 (a) are manually specified on the screen, so that a transformed fundus image 1701 (c) is generated and displayed. Similarly, for a fundus image 1701 (b) for the left eye, the coordinates of a macular region 301 (b) and an optic disc region 302 (b) are manually specified on the screen, so that a transformed fundus image 1701 (d) is generated and displayed. Alternatively, in specifying the macular region 301 and the optic disc region 302, an automatic selection button (not illustrated) may be pressed to automatically detect and specify the macular region 301 and the optic disc region 302. Furthermore, the information about the macular region 301 and the optic disc region 302 specified by the operator for the fundus image of either the left or right eye may be used to automatically detect and specify the macular region 301 and the optic disc region 302 in the fundus image of the other eye. For example, the coordinates of the macular region 301 (a) and the optic disc region 302 (a) specified in the fundus image 1701 (a) for the right eye may be horizontally flipped and the resulting flipped coordinates may be applied to the fundus image 1701 (b) for the left eye, thus enabling specification of the macular region 301 (b) and the optic disc region 302 (b). The pre-transformation fundus images 1701 (a), 1701 (b) and the post-transformation fundus images 1701 (c), 1701 (d) may be displayed side-by-side as illustrated in FIG. 17, or may be displayed individually or may be switched and displayed one by one. The method for specifying the macular region 301 and the optic disc region 302 is not limited to the above; instead of the macular region 301 and the optic disc region 302, values used for transforming the fundus image, such as the straight line 305 or angles α1 and β1 described in conjunction with FIGS. 5A and 5B, may be specified as appropriate.

Further, the square grids 801 and 803 are superimposed on the transformed fundus images 1701 (c) and 1701 (d), respectively. For each of the square grids 801 and 803, comparisons may be performed between the vertically corresponding A and D rows and between the vertically corresponding B and C rows to evaluate symmetry, and the results may be displayed. In addition, for the square grids 801 and 803, symmetry between the fundus images of the left and right eyes may be evaluated by comparing the cells having the same reference numerals, and the results thereof may be displayed. The method for evaluating the symmetry of the fundus images of the left and right eyes may be the same as the evaluation method used for one eye or may be a different evaluation method. Alternatively, the concentric circular grids 802 and 804 may be used instead of the square grids 801 and 803.

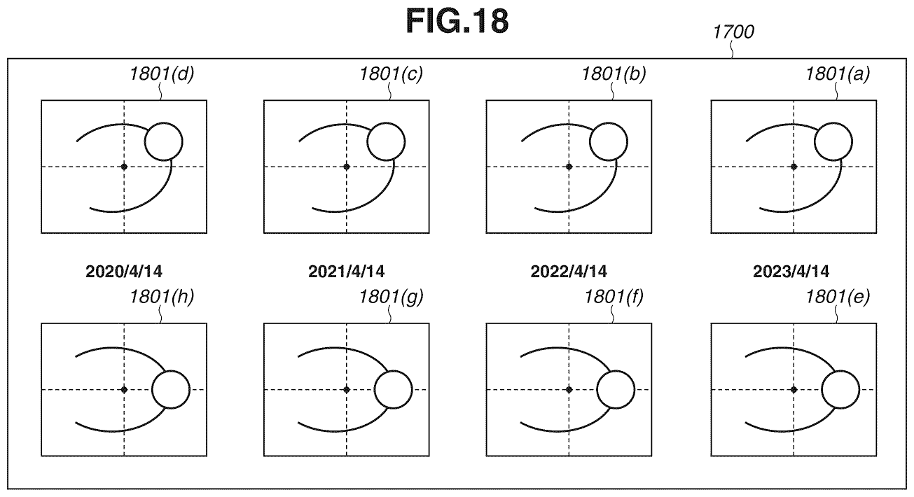

FIG. 18 illustrates an example of a GUI that displays, on the monitor 1700, results of analysis of fundus images of an eye under examination, which have been captured at different times for follow-up observation and then transformed. The fundus images captured at different times include, for example, a fundus image captured today, a fundus image captured one year ago, and a fundus image captured two years ago. Fundus images 1801 (a) to (d) and the corresponding transformed fundus images 1801 (e) to (h), respectively, are generated and displayed on the monitor 1700. The fundus images for display may be displayed individually or may be switched and displayed. In a case where a value used for transformation (e.g., the positions of the macular region 301 and the optic disc region 302) is changed for any of the fundus images 1801 (a) to (d), a similar value may be applied to the other fundus images so that all the fundus images are transformed collectively. For example, in a case where the positions of the macular region 301 and the optic disc region 302 in the fundus image 1801(a) are changed by the operator, the positions of the macular region 301 and the optic disc region 302 in each of the other fundus images (b) to (h) may also be changed in a similar manner. In such a case, the positions of the macular region 301 and the optic disc region 302 in the respective fundus images (b) to (h) may be obtained through image analysis. For example, a possible method involves identifying, based on the feature(s) of the macular region 301 in 1801 (a), a position having a similar feature(s) included in each of the fundus images (b) to (h) as the macular region 301 of the corresponding fundus image. Alternatively, transformation may be performed based on values individually set for each image. In addition, in comparing thickness difference information in follow-up observation, the result of comparing the thickness difference information about each of the pre-transformation fundus images 1801 (a) to (d) may be displayed, or the result of comparing the thickness difference information about each of the post-transformation fundus images 1801 (e) to (h) may be displayed.

### Second Embodiment

In the first embodiment, as illustrated in FIG. 5A, a case has been described in which the temporal regions 403 and 404 are not transformed and the nasal regions 501 and 502 are transformed, among the regions segmented by the straight lines 304, 305, and 401. In the first embodiment, the straight line 401 is set orthogonal to the straight line 304, based on the assumption that the region on the temporal side of the macular region 301 is bilaterally symmetrical. However, in actual fundus images, the region on the temporal side of the straight line 401 is not necessarily bilaterally symmetrical due to individual differences and other factors.

To address this, in the present embodiment, the first embodiment is partially modified. The information processing apparatus 100 according to a second embodiment has a configuration similar to that illustrated in FIG. 1. Thus, the same reference numerals are used and the description thereof will be omitted. In addition, the information processing apparatus 100 according to the present embodiment performs a process of the flowchart similar to that illustrated in FIG. 2, so that the description thereof will be provided using the same reference numerals. Hereinafter, operations of the control unit 102 according to the present embodiment will be described mainly focusing on the differences from the first embodiment.

### [Step S22: Thickness Map Transformation]

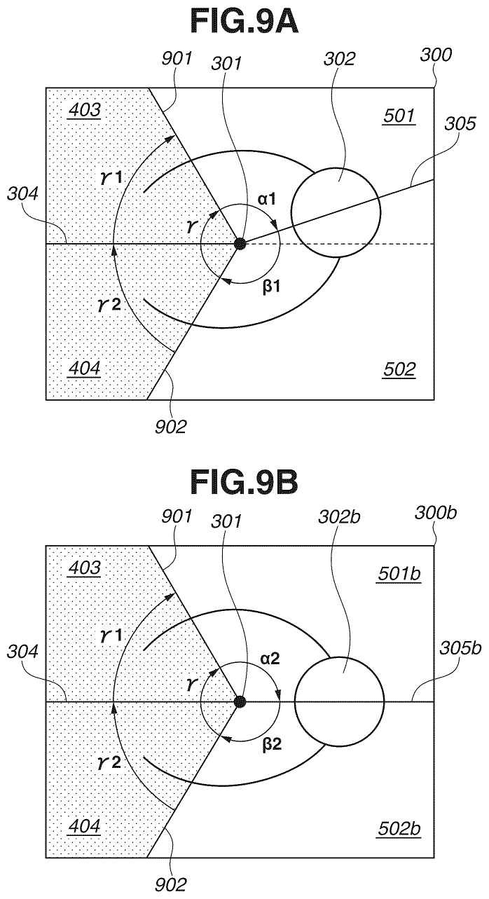

In step S22, the input fundus image is transformed. A specific transformation method will now be described with reference to FIGS. 9A and 9B. FIG. 9A illustrates a substantially horizontal straight line 304 with respect to a macular region 301 in a fundus image 300, and a straight line 305 connecting the macular region 301 and an optic disc region 302. From the macular region 301, straight lines 901 and 902 having certain angles γ1 and γ2 with respect to the straight line 304 are extended, and the regions segmented by the straight lines 304, 305, 901, and 902 are defined as follows: the superior temporal region as a region 403, the inferior temporal region as a region 404, the superior nasal region as a region 501, and the inferior nasal region as region 502. Here, the angle formed at the macular region 301 by the corner of the region 501 is defined as α1, the angle formed at the macular region 301 by the region 502 is defined as β1, and the angle formed at the macular region 301 by the corner of the combined region of regions 403 and 404 is defined as γ=γ1+γ2. Here, as in the first embodiment, the regions 403 and 404 of the fundus image 300 are not transformed, and the regions 501 and 502 can be transformed so that the straight line 305 matches the dashed portion of the straight line 304. The image after transformation is illustrated in FIG. 9B. The straight line 901 is an example of a third straight line, and the straight line 902 is an example of a fourth straight line.

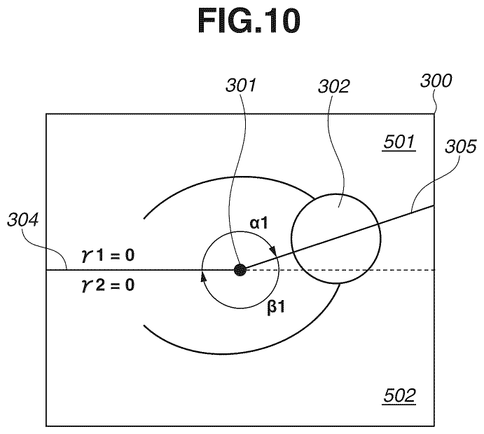

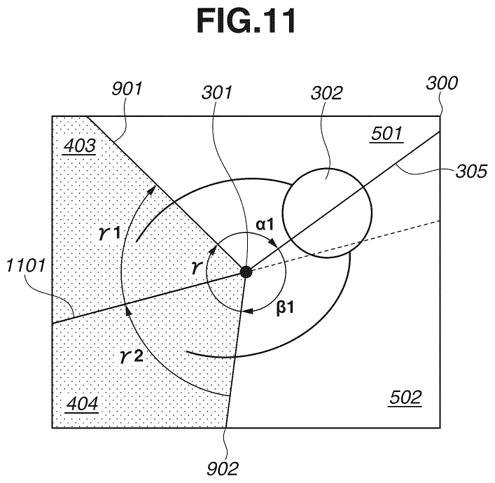

The angles γ1 and γ2, which are certain angles, may be manually set, or the angles γ1 and γ2 may be automatically set so that an index indicating symmetry with respect to the straight line 304 becomes the highest. The index indicating symmetry may be obtained, for example, from a difference in a thickness map at an equal distance in a direction perpendicular to a reference straight line. As illustrated in FIG. 10, the angles γ1 and γ2, which are certain angles, may be set to zero. Further, as illustrated in FIG. 11, a straight line 1101 having a certain inclination from the macular region 301 may be used instead of the straight line 304. The straight line 1101 may be manually set, or may be automatically set so that the index indicating symmetry with respect to the straight line 1101 becomes the highest. In FIG. 11, where the portion of the straight line 1101 on the temporal side of the macular region 301 is illustrated as a solid line and the nasal side as a dashed line, the regions 501 and 502 can be transformed so that the straight line 305 matches the dashed portion of the straight line 1101. Alternatively, the entire fundus image 300 may be rotated in advance so that the straight line 1101 becomes substantially horizontal (matches the straight line 304) before transformation.

In a case where the values of γ1 and γ2 differ, the temporal regions 403 and 404 may be transformed so that the straight line 1101 is rotated to satisfy γ1=γ2=γ/2, or the regions 403, 404, 501, and 502 may be transformed so that the straight lines 901 and 902 are rotated. In a case where the regions are transformed so that the straight lines 901 and 902 rotate, the portions of the nasal regions 501 and 502 other than the vicinity of the straight lines 901 and 902 may remain untransformed, or the nasal regions 501 and 502 may be transformed by compressing (reducing in size) or stretching (enlarging) the nasal regions 501 and 502 in the rotational direction, while maintaining the temporal regions 403 and 404 untransformed. Alternatively, some or all of the temporal regions 403 and 404 and the nasal regions 501 and 502, in whole or in part, may be transformed by compressing (reducing in size) or stretching (enlarging) them in the rotational direction. It is not always necessary for γ1 and γ2 to be equal. The transformation process may be applied even if γ1=γ2 before transformation, and it is also acceptable if γ1≠γ2.

The respective lines described in conjunction with FIG. 11 are not necessarily required to pass through the macular region 301 and the optic disc region 302, and may be freely specified on the fundus image or based on coordinates. The angles formed between the respective lines may also be freely set.

In a case where the thickness map transformation process is applied to fundus images of right and left eyes, the angles γ1 and γ2 and the straight line 1101 may be individually set for each fundus image, or may be linked between the left and right eyes. In a case where a plurality of fundus images captured by different apparatuses is displayed or where a plurality of fundus images is displayed in follow-up observations, if any of the angles α1, β1, γ1, γ2, the straight lines 305, 901, 902, 1101, and the like are changed for one fundus image, the other fundus images may be changed in coordination. Furthermore, in a case where the operator switches the layer to be targeted for generation of a thickness map or changes the type of map to be displayed, certain angles and lines may retain the same values as those before the map change, or different values may be retained for each map type. Alternatively, the values applied to a certain fundus image may also be applied to another fundus image to perform transformation. Additionally, these images may be combined, switched, or superimposed for display as desired.

### Third Embodiment

In the first and second embodiments, a method has been described in which the square grid 801 or the concentric circular grid 802 is superimposed on a transformed fundus image and a thickness map. In the present embodiment, a method will now be described in which a grid is superimposed on a fundus image and/or a thickness map prior to transformation. This method partially modifies the first and second embodiments.

The information processing apparatus 100 according to the present embodiment has a configuration similar to that illustrated in FIG. 1. Thus, the same reference numerals are used and the description thereof will be omitted. In addition, the information processing apparatus 100 according to the present embodiment performs a process of the flowchart similar to that illustrated in FIG. 2, so that the description thereof will be provided using the same reference numerals. Operations of the control unit 102 according to the present embodiment will be described below, focusing on differences from the first embodiment.

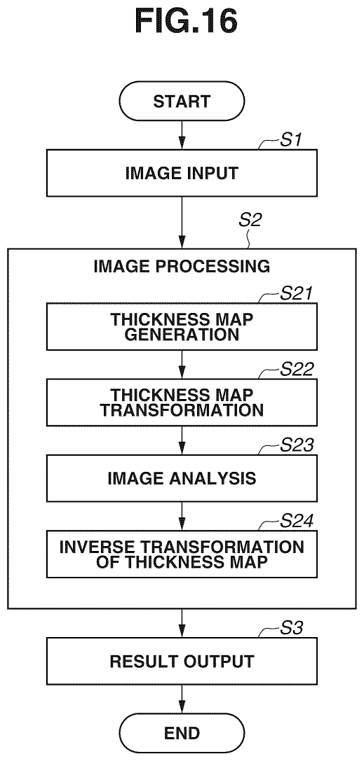

In FIG. 16, step S24 (inverse transformation of thickness map) is added after step S23 in the flowchart in FIG. 2.

### [Step S24: Inverse Transformation of Thickness Map]

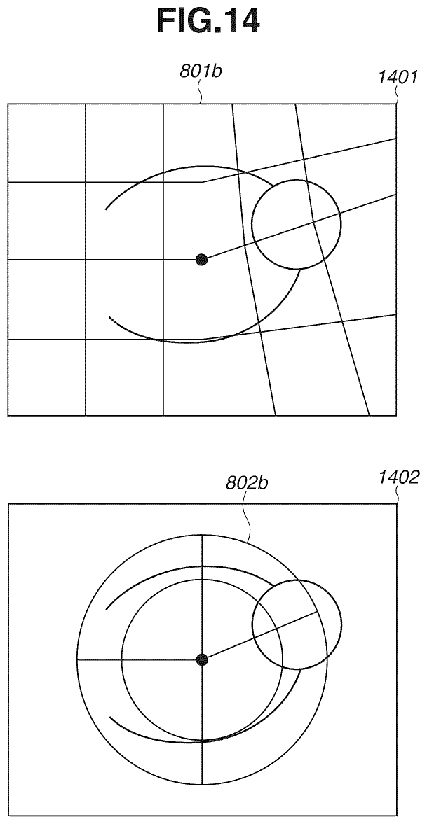

In step S24, the thickness map obtained through the transformation in step S22 is subjected to an inverse transformation so as to return to the state before the transformation. At this time, the analysis results obtained in step S23 are also subjected to the inverse transformation. Specifically, the fundus images 1301 and 1302, illustrated in FIG. 13, with the grids superimposed thereon are transformed through the inverse transformation into fundus images 1401 and 1402 illustrated in FIG. 14, and the superimposed grids 801 and 802 are transformed into grids 801b and 802b.

In the present embodiment, for ease of description, a method has been described in which a once-transformed fundus image is returned to the original fundus image through inverse transformation. However, the fundus image may remain untransformed, and the transformed grids 801b and 802b may be directly superimposed on the original fundus image.

### Fourth Embodiment

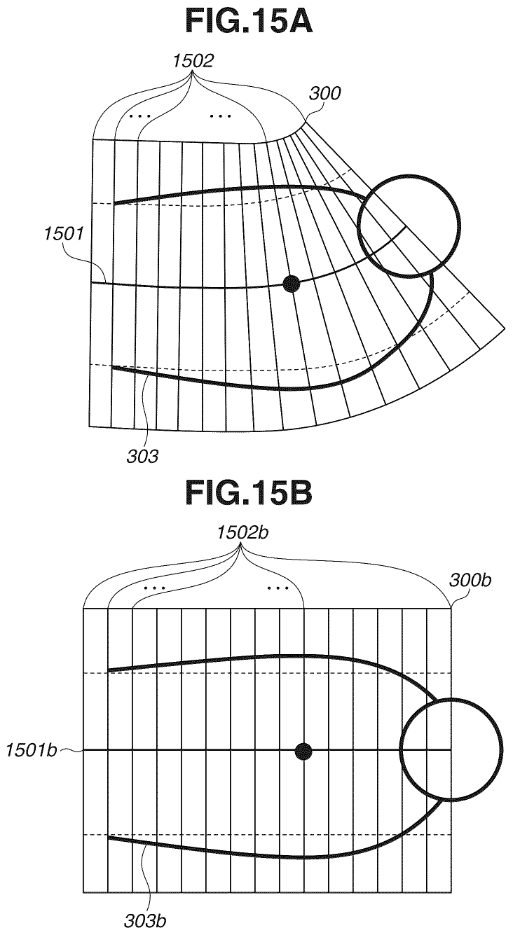

In the first and second embodiments, as illustrated in FIG. 3, a method has been described in which the straight lines 304 and 305 are set to straight lines, and an image segmented into temporal and nasal regions is transformed. Here, as illustrated in FIG. 15A, a fundus image may have symmetry with respect to a curve 1501 rather than a straight line. In such a case, segmentation into only temporal and nasal regions may be insufficient.

In the present embodiment, the first and second embodiments are partially modified to address the foregoing. The information processing apparatus 100 according to the present embodiment has a configuration similar to that illustrated in FIG. 1. Thus, the same reference numerals are used and the description thereof will be omitted. In addition, the information processing apparatus 100 according to the present embodiment performs a process of the flowchart similar to that illustrated in FIG. 2, so that the description thereof will be provided using the same reference numerals. Hereinafter, operations of the control unit 102 according to the present embodiment will be described mainly focusing on the differences from the first embodiment. [Step S22: Thickness Map Transformation]

In step S22, the input fundus image is transformed. A specific transformation method will now be described with reference to FIGS. 15A and 15B. FIGS. 15A and 15B illustrate an example in which the fundus image 300 is transformed so that the curve 1501 becomes a straight line 1501b, thus generating a fundus image 300b.

FIG. 15A illustrates auxiliary straight lines 1502 orthogonal to the curve 1501 at any points on the curve 1501. If the curve 1501 is transformed into the straight line 1501b, the auxiliary straight lines 1502 are transformed into auxiliary straight lines 1502b orthogonal to the straight line 1501b. Similarly, any points on the auxiliary straight line 1502 are transformed onto those on the auxiliary straight line 1502b, so that a blood vessel 303 is transformed into a blood vessel 303b.

Here, the curve 1501, which is a certain curve, may be set manually. Alternatively, the curve 1501 may be automatically set so that an index of symmetry with respect to the straight line 1501b, which is obtained after transformation, becomes the highest. In addition, in the present embodiment, a method has been described in which the auxiliary straight lines 1502 are set to transform the fundus image; however, it is also acceptable to segment the fundus image into a plurality of regions as illustrated in FIG. 15A and transform each region individually.

While embodiments of the present disclosure have been described above, it should be understood that the present disclosure is not limited to these embodiments, and various modifications and changes can be made within the scope of the gist of the present disclosure.

According to the present disclosure, the symmetry of a layer thickness of a region on a nasal side and the symmetry of a layer thickness of a region on a temporal side can be appropriately evaluated.

### OTHER EMBODIMENTS

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The present disclosure is not limited to the above-described embodiments, and various changes and modifications can be made without departing from the spirit and scope of the present invention. Accordingly, the following claims are appended to describe the scope of the present disclosure.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

1. An information processing apparatus, comprising:
a transformation unit configured to transform a first region and a second region in a fundus image so that an angle formed between a first straight line and a second straight line approaches 180 degrees, the first and second regions being defined by the first straight line and the second straight line, the second region being larger than the first region, the first straight line passing through a first feature portion related to a macula in the fundus image and being located on a temporal side, the second straight line passing through the first feature portion and a second feature portion related to an optic disc in the fundus image and being located on a nasal side.

2. The information processing apparatus according to claim 1, wherein the transformation unit transforms the first region and the second region so that the second straight line rotates about the first feature portion as a fulcrum and an angle formed between the first straight line and the second straight line approaches 180 degrees.

3. The information processing apparatus according to claim 1, wherein the transformation unit transforms the first region and the second region by enlarging the first region and reducing a size of the second region so that an angle formed between the first straight line and the second straight line approaches 180 degrees.

4. The information processing apparatus according to claim 1, wherein the transformation unit transforms a region, in the first region, on the nasal side of a third straight line passing through the first feature portion and located in the first region without transforming a region, in the first region, on the temporal side of the third straight line, and transforms a region, in the second region, on the nasal side of a fourth straight line passing through the first feature portion and located in the second region without transforming a region, in the second region, on the temporal side of the fourth straight line.

5. The information processing apparatus according to claim 1, wherein the transformation unit transforms a region, in the first region, on the temporal side of a third straight line passing through the first feature portion and located in the first region without transforming a region, in the first region, on the nasal side of the third straight line, and transforms a region, in the second region, on the temporal side of a fourth straight line passing through the first feature portion and located in the second region without transforming a region, in the second region, on the nasal side of the fourth straight line.

6. The information processing apparatus according to claim 1, further comprising an image generation unit configured to generate a second fundus image in which a grid defining a plurality of evaluation regions and evaluation indices in the evaluation regions are superimposed on the fundus image obtained through transformation performed by the transformation unit.

7. The information processing apparatus according to claim 6, wherein the transformation unit inversely transforms the second fundus image so that an angle formed between the first straight line and the second straight line in the second fundus image approaches an angle formed between the first straight line and the second straight line in the fundus image.

8. The information processing apparatus according to claim 6, wherein each of the evaluation indices is at least one of an average value, a median value, a standard deviation, and a variance of thicknesses of a fundus included in a corresponding evaluation region.

9. The information processing apparatus according to claim 6, wherein the image generation unit generates a third fundus image in which difference information about a difference in the evaluation indices of evaluation regions that are in a symmetrical relationship with respect to the first straight line, and difference information about a difference in the evaluation indices of evaluation regions that are in a symmetrical relationship with respect to the second straight line, are further superimposed.

10. The information processing apparatus according to claim 1, wherein the fundus image is a thickness map.

11. The information processing apparatus according to claim 1, wherein at least one of the first feature portion and the second feature portion is specified based on an instruction from an operator or based on a result of analysis performed on the fundus image.

12. The information processing apparatus according to claim 1, wherein, in a case where at least one of the first feature portion and the second feature portion is specified based on an instruction from an operator in a fundus image of either a right eye or a left eye of a subject, at least one of the first feature portion and the second feature portion is specified in a fundus image of the other eye of the subject using information about the specification.

13. The information processing apparatus according to claim 1, further comprising a display control unit configured to perform control to cause a display unit to display at least one of the fundus image and a fundus image obtained through transformation performed by the transformation unit.

14. The information processing apparatus according to claim 13, wherein the display control unit performs control to cause the display unit to display, side by side, a fundus image of a right eye obtained through transformation performed by the transformation unit and a fundus image of a left eye obtained through transformation performed by the transformation unit.

15. The information processing apparatus according to claim 13, wherein the display control unit performs control to cause the display unit to display, side by side, a plurality of first fundus images obtained by imaging an eye under examination at different times and a plurality of second fundus images obtained by the transformation unit transforming the plurality of first fundus images.

16. The information processing apparatus according to claim 14, wherein, in a case where at least one of the first feature portion and the second feature portion is specified based on an instruction from an operator in any one of the fundus images displayed on the display unit, the transformation unit specifies at least one of the first feature portion and the second feature portion in the other fundus image using information about the specification.

17. A method comprising:
transforming a first region and a second region in a fundus image so that an angle formed between a first straight line and a second straight line approaches 180 degrees, the first and second regions being defined by the first straight line and the second straight line, the second region being larger than the first region, the first straight line passing through a first feature portion related to a macula in the fundus image and being located on a temporal side, the second straight line passing through the first feature portion and a second feature portion related to an optic disc in the fundus image and being located on a nasal side.

18. A non-transitory storage medium storing a program for causing a computer to execute the transformation method according to claim 17.

19. An information processing system comprising:
an imaging apparatus configured to capture the fundus image; and
the information processing apparatus according to claim 1 communicably connected to the imaging apparatus.
